(19) United States
(12) Patent Application Publication
(10) Pub. No.: US 2008/0026416 A1
(43) Pub. Date: Jan. 31, 2008

(54) Model Taste Cells and Methods of Use for Identifying Modulators of Taste Sensation

(76) Inventors: Harish Radhakrishna, Tucker, GA (US); Michael D. Brown, Lilburn, GA (US)

Correspondence Address: SUTHERLAND ASBILL & BRENNAN LLP, 999 PEACHTREE STREET, N.E., ATLANTA, GA, 30309, US

(21) Appl. No.: 11/829,468

(22) Filed: Jul. 27, 2007

Related U.S. Application Data
(60) Provisional application No. 60/820,490, filed on Jul. 27, 2006.

(51) Int. Cl. C12Q 1/02; C12N 5/08; C12Q 1/34; C12Q 1/48

(52) U.S. Cl. 435/15; 435/18; 435/29; 435/366

(57) ABSTRACT

The present invention provides model taste cells that naturally or recombinantly express taste receptors and relevant cellular proteins and/or molecules useful for taste signal transduction. The present invention further provides methods of use for these model taste cells for screening for compounds that modulate sweet and/or other taste signal transduction. Compositions comprising the compounds/modulators identified using the model taste cells are also provided. In preferred embodiments, the model taste cells are derived from human HuTu-80 enteroendocrine cells, and derivative cells thereof.

### CROSS REFERENCE TO RELATED APPLICATIONS

This application claims the priority benefit of U.S. Provisional Application Ser. No. 60/820,490, filed Jul. 27, 2006, the entire contents of which are hereby incorporated by reference.

### FIELD OF THE INVENTION

The present invention relates to cells that endogenously and/or naturally express one or more signaling proteins and relevant cellular machinery necessary for taste signal transduction, and methods for using these cells for identifying compounds/modulators for modulating taste signaling. Particularly, the present invention relates to methods for using HuTu-80 human enteroendocrine cells, and derivative cells thereof, that naturally express taste receptors and the relevant cellular signal transduction machinery to identify taste modulatory compounds.

### BACKGROUND OF INVENTION

Taste cells are assembled into taste buds on the tongue surface (Lindemann, 1996, Physol. Rev. 76:718-66). Two families of GPCRs have been identified in taste cells: the T1R family of GPCRs that mediates sweet and umami tastes, and the T2R family of GPCRs that mediate bitter tastes (Nelson et al., 2001, Cell 106: 381-90; Nelson et al., 2002, Nature 416:199-202; Li et al., 2002, Proc. Natl. Acad. Sci. USA 99; 4692-96; Zhao et al., 2003, Cell 115:255-66; Adler et al., 2000, Cell 100: 693-702; Chandrashekar et al., 2000, Cell 100: 703-11; Bufe et al., 2002, Nat. Cenet. 32: 397-401). Signaling downstream of all of these receptors has been shown to depend on the key effector enzyme of sweet, umami and bitter taste transduction, phospholipase C subtype β2 (PLCβ2) and the trp channel subtype m5 (Zhang et al., 2003, Cell 112: 293-301).

The sense of taste can be divided into five primary sensations: bitter, salty, sour, sweet and umami (i.e., the response to salts of glutamic acid). Different taste modalities appear to function by different mechanisms. For instance, sweet taste seems to be mediated via G-protein-coupled T1R receptors that are heterodimers of subunits T1R2 and T1R3; bitter taste seems to be mediated by one or more G-protein coupled T2R receptors; and umami taste seems to be mediated by heterodimers of T1R1 and T1R3 (Zhao et al., 2003, Cell, 115, 255-266) and perhaps also by modified versions of metabotropic glutamate receptors known as mGluR4 (Chaudhari and Roper, 199, Ann. NY Acad. Sci., 855, 398-406).

Gustducin is a taste-selective G protein (McLaughlin et al., 1992, Nature, 357, 563-69). Activation of gustducin triggers a cascade of intracellular reactions: activation of phosphodiesterase; degradation of 3′,5′-cyclic adenosine monophosphate (cAMP) and 3′,5′-cyclic guanosine monophosphate (cGMP); and the closing of cyclic nucleotide gated cation channels that leads to depolarization of the cell. Gustducin is about 80%-90% homologous to transducin, which has also been immunocytochemically localized to taste buds, and has been implicated in taste signal transduction by activation of a taste-specific PDL activity (Ruiz-Avila et al., 1995, Nature, 376, 80-85). Gustducin has been implicated in vivo in transducing responses to bitter and sweet compounds (Wong et al., 1996, Nature, 381, 796-800).

The gustatory system has been selected to detect sweet and/or bitter substances (Herness & Gilbertson, 1999, Annu Rev Physiol 61:873-900; Hoefer & Drenckhahn, 1998, Histochem Cell Biol. 110: 303-309). Outside the tongue, expression of the α-subunit of gustducin (Gagust) has been also localized to gastric (Hoefer et al., 1996, Proc. Natl. Acad. Sci USA 93: 6631-34; Wu et al., 2002, Proc. Natl. Acad. Sci USA 99: 2392-97), and pancreatic cells (Hoefer & Drenckhahn, 1998, Histochem Cell Biol. 110: 303-309), suggesting that a taste-sensing mechanism may also exist in the GI tract (Wu et al., 2005, Physiol Genomics 22: 139-149; Wu et al., 2002, Proc. Natl. Acad. Sci USA 99: 2392-97).

Various taste receptors have been found in enteroendocrine cell lines, such as STC and NCI cell lines. For instance, UCLA researchers have isolated and identified via single cell cloning a derivatized STC-1 line that homogenously expresses a panel of T2R receptors and alpha subunits of G proteins implicated in intracellular taste signal transduction, namely Gagust and Gat-2, and suggest that derivatized STC-1 may be used to assay for compounds that activate T2R receptors that may modulate appetite (WO03031604, see also UCLA Technology Available For Licensing: EXPRESSION OF GASTROINTESTINAL CHEMSENSORY RECEPTORS IN DERIVATIZED MOUSE STC-1 CELL LINES). Human intestinal endocrine cell line NCI-H716 has also been demonstrated to contain one or more taste signaling proteins, and has been used to screen and identify taste modulators for taste sensation (U.S. Publication Nos. 20050244810, 20050177886). In addition, it has been reported that, HuTu-80 cells also express α-gustducin, some gastric peptide hormones, a large number of T2R bitter receptors, and T1R3 receptor only (Rozengurt et al., 2006, Am J. Physiol Gastrointest Liver Physiol 291: G792-802). However, Rozengurt et al. was unable to show that HuTu-80 cells express T1R2 sweet receptors, and provide any data to support that HuTu-80 responds to any sweeteners.

Over the past decade substantial efforts have been directed to the development of various agents that interact with taste receptors to mimic or block natural taste stimulants (Cagan, 1989, Ed., Neural Mechanisms in Taste, Chapter 4, CRC Press, Inc., Boca Raton, Fla.). However, development of new agents that mimic or block the four basic tastes has been limited by a lack of knowledge of the taste cell proteins responsible for transducing the taste modalities. There continues to exist a need in the art for new products and methods that are involved in or affect taste detection and/or transduction. Finding human-model experimental systems to study taste detection and transduction would aid in our understanding of the molecular biology and biochemistry of taste. Such a model system would be useful for screening for novel sweeteners, enhancers of desirable flavors, or blockers of undesirable flavors.

### SUMMARY OF THE INVENTION

The present invention provides an alternate and abundant source of taste-sensing cells (“the model taste cells”), which endogenously and/or naturally express the taste receptors and associated signaling proteins and the relevant cellular machinery, for use in high throughput screening assays to identify compounds and/or modulators for taste signaling. In one preferred embodiment, the present invention provides the model taste cells that are derived from human HuTu-80 enteroendocrine cells, preferably the parental HuTu-80 cell line (ATCC: HTB-40™), and more preferably an enriched sweet-sensitive subcloned and/or modified cell line derived from parental HuTu-80 cells (ATCC: HTB-40™). The HuTu-80 cells and any enriched subcloned and/or modified cells of the present invention exhibit some taste cell functionality, and comprise endogenously and/or naturally expressed one or more signaling proteins and the relevant cellular machinery necessary for taste signal transduction.

In yet another preferred embodiment, the model taste cells of the present invention comprise endogenously and/or naturally expressed sweetener receptors and/or its homo- or hetero-oligomers, and one or more other proteins and the relevant cellular machinery for sweet taste signaling. In yet another preferred embodiment, the model taste cells of the present invention comprise endogenously and/or naturally expressed umami receptors or bitter receptors. In yet another preferred embodiment, the model taste cells of the present invention comprise naturally expressed G proteins, such as Gα proteins. In yet another preferred embodiment, the model taste cells of the present invention comprise naturally expressed regulator G protein signaling (RGS) proteins. In yet another preferred embodiment, the model taste cells of the present invention comprise naturally expressed effectors for taste signal transduction. The present invention further provides methods of producing the model taste cells from human HuTu-80 endocrine cells, and derivative cells thereof.

The present invention also provides methods for using the model taste cells from HuTu-80 endocrine cells, and derivative cells thereof, for identifying compounds and/or modulators for modulating taste signaling. In one preferred embodiment, the present invention provides methods of screening for a plurality of compounds that modulate taste signaling using the model taste cells of the present invention. Such methods may also comprise isolating and purifying one or more proteins of interest necessary for taste signal transduction from the model taste cells of the prevent invention. The methods further comprise determining effects of test compounds on the purified proteins of interest or their interactions with other proteins and/or the relevant cellular machinery in taste signal transduction using a variety of cell-based assays; identifying the test compound that modulate the purified proteins of interest, or their interactions with other proteins and the relevant cellular machinery in taste signal transduction based on said cell-based assays; and validating the compound in modulating the taste signaling in the model taste cells.

In one preferred embodiment, the proteins of interest that are isolated and purified from the model taste cells of the present invention comprise one or more taste receptors (e.g. T1R2 and/or T1R3), G proteins, RGS proteins, effectors, and/or further relevant cellular machinery useful for taste signal transduction. In yet another preferred embodiment, the proteins of interest are taste receptors comprising sweetener receptors and their homo- or hetero-oligomers; bitter receptors and their homo- or hetero-oligomers, or umami receptors. In yet another preferred embodiment, the proteins of interest are G proteins comprising Gα proteins selected from a group consisting of Gαi proteins, α-gustducin, Gαi2, and others. In yet another preferred embodiment, the proteins of interest are RGS proteins comprising GAIP, RGSz1, RGS1, RGS2, RGS3, RGS4, RGS5, RGS6, RGS7, RGS8, RGS9, RGS10, RGS11, RGS12, RGS13, RGS14, RGS16, RGS17, RGS21, D-AKAP1, p115RhoGEF, PDZ-RhoGEF, bRET-RGS, Axin, and mCONDUCTTN. In yet another preferred embodiment, the proteins of interest are effectors comprising phospholipase C (PLC), cAMP, cGMP, IP3, calcium (Ca²⁺) and other second messengers.

In yet another preferred embodiment, the present invention provides methods of screening for a plurality of compounds for enhancing sweet taste. Such methods comprise 1) providing the model taste cells of the present invention, wherein the taste cells endogenously and/or naturally express taste receptors and one or more other proteins and/or the relevant cellular machinery necessary for taste signaling; 2) contacting the model taste cells with a tastant alone, or in combination with test compounds; 3) determining effects of test compounds on the model taste cells using cell-based assays to monitor one or more of a) changes in intracellular second messengers (e.g., cAMP, cGMP, calcium, phophoinositides); b) changes in protein kinase activity (e.g., ERK, PKC, Src, EGFR, etc.); c) changes in secretion of gastrointestinal peptides (e.g., peptide YY (PYY), glucagon, glucagon-like peptide-1 (GLP-1), gastric insulinotropic peptide (GIP), etc.) and d) changes in neurotransmitter secretion by model taste cell; 4) identifying a compound that provide the changes as described above in 3); and 5) validating an efficacy of the identified compound in human sensory taste tests for enhancing taste by the tastant. As used herein, the tastants include sweeteners, bitters, and other taste modulators. As used herein, the sweeteners include, but are not limited to, carbohydrate sweeteners, synthetic high-potency sweeteners, natural high-potency sweeteners, polyols, and amino acids.

In yet another preferred embodiment, the present invention provides methods of screening for a plurality of compounds for enhancing human taste. Such methods comprise providing the model taste cells of the present invention, wherein the taste cells naturally express RGS proteins and one or more other proteins and/or the relevant cellular machinery necessary for taste signaling, such as Gα proteins; identifying compounds that inhibit RGS protein activity (RGS protein inhibitors); determining a taste signal activated by a taste receptor with a tastant alone, and in combination with the compounds (RGS protein inhibitors); and identifying compounds (RGS protein inhibitors) that increase the taste signaling of said tastant. In one preferred embodiment, the RGS protein is RGS21 protein.

Moreover, the present invention provides methods to validate the effects of identified compounds and/or modulators using the model taste cells of the present invention on human sweet taste, as well as umami and bitter taste. In one preferred embodiment, the present invention provides a comparison of the perceived sweetness of a test sweetener tasted by itself to that of a combination of a test sweetener and the identified modulatory compounds.

### DETAILED DESCRIPTION OF THE INVENTION

The present invention provides the model taste cells and methods for using these cells to screen and identify modulators for sweet taste signaling, as well as for umami and bitter taste signaling. The model taste cells of the present invention comprise human HuTu-80 endocrine cells and any subclones and/or modified cells derived from these cells.

The present invention also provides the model taste cells and methods of producing the model taste cells from human HuTu-80 endocrine cells (Rozengurt et al., 2006, Am J Physiol Gastrointest Liver Physiol 291:792-802, the entire contents of which is incorporated by reference herewith), and/or derivative cells thereof The present invention further provides methods of using these model taste cells for screening and identifying compounds for modulating taste signal transduction, including sweet taste signaling, umami taste signaling, and bitter taste signaling.

As used herein, the term “taste bud cells” or “taste cells” are used interchangeably and include neuroepithelial cells that are organized into groups to form taste buds of the tongue, e.g., foliate, fungiform, and circumvallate cells (Roper et al., 1989, Ann. Rev. Neurosci. 12:329-353). Taste cells are also found in the palate and other tissues, such as the esophagus, intestine, and the stomach.

As used herein, the term “model taste cells” refers to cell lines that are capable of producing taste cells that endogenously and/or naturally express one or more signaling proteins and/or the relevant cellular machinery useful for taste signal transduction. As used herein, the term “model taste cells” also refers to cells including, but not limited to, human HuTu-80 endocrine cells, and derivative cells thereof, including any subcloned and/or modified cells derived from HuTu-80 endocrine cells.

As used herein, the terms “express” or “expression” are used to refer to the cellular production of proteins of interest by genomic or recombinant nucleic acid sequences under either naturally occurring conditions or in response to exogenous signals or promoters. As used herein, “express” or “expression” includes the process by which polynucleotides are transcribed into RNA and/or translated into polypeptides in a host cell. If the polynucleotide is derived from genomic DNA, expression may include splicing of the RNA, if an appropriate eukaryotic host cell is selected. Regulatory elements required for expression include natural or recombinant promoter sequences to bind RNA polymerase and transcription initiation sequences for ribosome binding. For example, a bacterial expression vector includes a promoter such as the lac promoter and for transcription initiation the Shine-Dalgamo sequence and the start codon AUG. Similarly, a eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described below for constructing vectors in general. As used herein, the term “vector” includes a self-replicating nucleic acid molecule that transfers an inserted polynucleotide into and/or between host cells. The term is intended to include vectors that function primarily for insertion of a nucleic acid molecule into a cell, replication vectors that function primarily for the replication of nucleic acid and expression vectors that function for transcription and/or translation of the DNA or RNA. Also intended are vectors that provide more than one of the above function.

As used herein, a “host cell” is intended to include any individual cell or cell culture which can be, or has been, a carrier of endogenous polynucleotides and/or polypeptides or a recipient for vectors for the incorporation of exogenous polynucleotides and/or polypeptides. It is also intended to include progeny of a single cell. The progeny may not necessarily be completely identical (in morphology or in genomic or total DNA complement) to the original parent cell due to natural, accidental, or deliberate mutation. The cells may be prokaryotic or eukaryotic, and include but are not limited to bacterial cells, yeast cells, insect cells, animal cells, and mammalian cells, including but not limited to murine, rat, simian or human cells. Therefore, as used herein, a “host cell” also includes genetically modified cells. The term “genetically modified cells” includes cells containing and/or expressing a foreign or exogenous gene or polynucleotide sequence which in turn modifies the genotype or phenotype of the cell or its progeny. “Genetically modified” also includes a cell containing or expressing a gene or polynucleotide sequence which has been introduced into the cell. For example, in this embodiment, a genetically modified cell has had introduced a gene which gene is also exogenous to the cell. The term “genetically modified” also includes any addition, deletion, or disruption to a cellos endogenous nucleotides. As used herein, a “host cell” also includes naturally occurring taste bud cells and taste bud cell precursor cells. As used herein, the “host cells” also refers to the model taste cells including, but are not limited to, human HuTu-80 enteroendocrine cells, and derivative cells thereof

The present invention further provides methods of screening for a plurality of compounds that modulate taste signaling using the model taste cells as defined above. Such methods can comprise isolating and purifying one or more proteins of interest necessary for taste signal transduction from the model taste cells of the present invention; determining effects of test compounds on the purified proteins of interest or their interactions with other proteins in taste signal transduction using variety of cell-free and cell-based assays; identifying the test compound that modulates the purified proteins of interest, or their interactions with other proteins in taste signal transduction based on said cell-based assays; and validating the compound in modulating the taste signaling.

As used herein, the term “compounds” or “test compounds” is used interchangeably, and preferably refers to small molecules or bioactive agents. Bioactive agents include, but are not limited to, naturally-occurring or synthetic compounds or molecules (“biomolecules”) having bioactivity in mammals, as well as proteins, peptides, oligopeptides, polysaccharides, nucleotides and polynucleotides. Preferably, the bioactive agent is a protein, polynucleotide or biomolecule. One skilled in the art will appreciate that the nature of the test compound may vary depending on the nature of the protein and/or molecules of interest as defined below. The test compounds of the present invention may be obtained from any available source, including systematic libraries of natural and/or synthetic compounds.

In general, methods and compositions for screening for protein inhibitors or activators and in vitro and/or in vivo protein-to-protein and protein-to-ligand binding studies are known in the art, and may be used in combination with the methods of the invention. In one embodiment, the present invention provide a method of screening for test compounds capable of modulating the binding of a protein of interest and a corresponding Gα protein in the model taste cells of the present invention, by isolating and purifying the protein of interest and the Gα protein from the model taste cells, and combining the test compound, the purified protein of interest, and the purified Gα protein together, and further determining whether binding of the protein of interest and Gα protein occurs and/or changes in the presence of the test compound. As discussed below, test compounds may be provided from a variety of libraries well known in the art.

In yet another embodiment, the present invention provides a screening assay using the model taste cells to detect a test compound's ability to bind to and module taste receptors. In yet another embodiment, the present invention provides a screening assay using the model taste cells to detect a test compounds' ability to inhibit the binding of RGS21 protein to Gα protein. In yet another embodiment, inhibitors/modulators of taste receptors and/or RGS21 proteins that modulate expression, activity or binding ability of these proteins are also provided using the model taste cells of the present invention.

As used herein, the terms “modulatory/modulation/modulator,” “inhibitory/inhibiting/inhibitors,” “activating/activators” including their various grammatical forms are used interchangeably to refer to modulating, inhibiting and/or activating protein molecules and/or the relevant cellular machineries, e.g., ligands, agonists, antagonists, and their homologs and mimetics, that are useful for taste signaling including effecting expression of genes or proteins, or fragments thereof comprising biologically active portion of molecules and/or cellular machineries of interest. Modulators include compounds that alter the interactions of genes or proteins, or fragment thereof comprising biological active portion, with their corresponding Gα proteins and other effectors and/or the relevant cellular machineries in taste signal transduction; and arresting, deactivating and desensitizing the expression levels of genes or proteins, or fragment thereof comprising biological active portion, of interest. Modulators can include genetically modified versions of genes or proteins of interest with altered activity, as well as naturally occurring and synthetic ligands, antagonists, agonists, small chemical molecules and the like. “Modulatory effect” refers to up-regulation, induction, stimulation, potentiation, attenuation, and/or relief of inhibition, as well as inhibition and/or down-regulation or suppression. Inhibitors are compounds that, e.g., bind to, partially or totally block stimulation, decrease, prevent, delay activation, inactivate, desensitize, or down regulate genes or proteins of interest, e.g., antagonists. Activators are compounds that, e.g., bind to, stimulate, increase, open, activate, facilitate, enhance activation, sensitize, or up regulate gene or proteins of interest, e.g., agonists.

As used herein, a “biologically active portion” of a protein of interest includes a fragment of a protein comprising amino acid sequences sufficiently homologous to, or derived from, the amino acid sequence of the protein, which include fewer amino acids than the full length protein, and exhibits at least one activity of the full-length protein. Typically a biologically active portion comprises a domain or motif with at least one activity of the protein. A biologically active portion of a protein can be a polypeptide which is, for example, 10, 25, 50, 100, 200 or more amino acids in length.

In yet another preferred embodiment, the model taste cells of the present invention comprise naturally expressed taste receptors including sweetener receptors, umami receptors, or bitter receptors, and/or its homo- or hetero-oligomers, and one or more other proteins and/or the relevant cellular machineries for sweet taste signaling. In yet another preferred embodiment, the model taste cells of the present invention comprise naturally expressed G proteins, such as Gα proteins. In yet another preferred embodiment, the model taste cells of the present invention comprise naturally expressed regulator G protein signaling (RGS) proteins. In yet another preferred embodiment, the model taste cells of the present invention comprise naturally expressed effectors for taste signal transduction. In yet another preferred embodiment, the model taste cells of the present invention comprising naturally expressed cellular machineries that are necessary for taste signaling.

As used herein, the term “taste receptors” refers to receptor proteins existing on the surface of taste cell membrane, that upon binding to their agonists and/or ligands activates taste signal transduction through a G protein coupled receptors (GPCRs) signal transduction pathway. The “taste receptors” refer to “sweetener receptors” including all members of T1R family of the GPCRs, now known or later described, that modulate sweet and/or umami taste signaling, including but not limited to putative T1Rs, homo-oligomers, such as T1R1/T1R1, T1R2/T1R2 and T1R3/T1R3; hetero-oligomers, such as T1R1/T1R3 and T1R2/T1R3, and their isoforms and homologs. The “taste receptors” also refer to “bitter receptors” including all members of T2R family of the GPCRs, now known or later described, that modulate bitter taste signaling, including not limited to putative T2Rs, homo-oligomers, hetero-oligomers, and their isoforms or homologs. Included in the invention are taste receptors which are at least 60% homologous, preferably 75% homologous, more preferably 85%, 90%, 95%, 96%, 97%, 98%, 99%, or more homologous, to a wild type T1R or T2R protein.

As used herein, the term “Gα” or “Ga proteins” includes all members of the Gαi class now known or later described, including but not limited to Gαi1-3, Gαz, Gαo, Gαs, Gαolf, Gαt, Gαq, Gα11-14, and Gα16. In certain embodiments, a Gα protein may contain one or more mutations, deletions or insertions. In such embodiments, the Gα. protein is at least 60% homologous, preferably 75% homologous, more preferably 85%, 90%, 95%, 96%, 97%, 98%, 99%, or more homologous, to a wild type Gα protein. As used herein, the term “corresponding and/or appropriate Gα protein” means a Gα protein which is capable of contacting an RGS protein of interest, e.g. RGS21 protein, in the cell, screening assay or system in use. Corresponding Gα proteins are also coupled to the GPCR and/or bound to GTP in the cell, screening assay or system in use such that the Gα protein is capable of contacting the GPCR and/or GTP, or is capable of transducing a signal in response to agonist binding to the GPCR. As used herein, the term “agonist binding to the GPCR” includes any molecule or agent which binds to GPCR and elicits a response.

As used herein, the term “RGS” or “RGS protein” includes regulators of G protein signaling and/or proteins now known, or later described, which are capable of inhibiting or binding to a Gαi class proteins or other Gα proteins. Such RGS proteins include, but are not limited to, GAIP, RGSz1, RGS1, RGS2, RGS3, RGS4, RGS5, RGS6, RGS7, RGS8, RGS9, RGS10, RGS11, RGS13, RGS14, RGS16, RGS17, RGS21, D-AKAP2, p115RhoGEF, PDZ-RhoGEF, bRET-RGS, Axin, and mCONDUCTIN, as well as any now known, or later described, isoforms or homologs. In addition, as used herein, the term “RGS protein” includes now known, or later described, proteins that contain a RGS core domain, including RGS-box domain, non-RGS-box domain or any other functional domains/motifs, with or without one or more mutations, deletions or insertions. In one preferred embodiment, the RGS protein refers to RGS21 protein, its isoforms or homologs. In yet another preferred embodiment, the RGS21 protein core domain is at least 60% homologous, preferably 75% homologous, more preferably 85%, 90%, 95%, 96%, 97%, 98%, 99% or more homologous, to a wild type RGS21 protein core domain. As used herein, the RGS21 protein core domain comprises biological active portion of the protein.

The present invention also provide methods of isolating and purifying genes and/or proteins of interest from the model taste cells of the present invention. As used herein, the term “isolated/isolating” or “purified/purifying” proteins, polypeptides, polynucleotides or molecules means removed from the environment in which they naturally-occur, or substantially free of cellular material, such as other contaminating proteins from the cell or tissue source from which the protein polynucleotide or molecule is derived, or substantially free from chemical precursors or other chemicals when chemically synthesized. The language “substantially free of cellular material” includes preparations separated from cellular components of the cells from which it is isolated or recombinantly produced or synthesized. In one embodiment, the language “substantially free of cellular material” includes preparations of a protein of interest having less than about 30% (by dry weight) of other proteins (also referred to herein as a “contaminating protein”), more preferably less than about 20%, still more preferably less than about 10%. and most preferably less than about 5% of other proteins. When the protein or polynucleotide is recombinantly produced, it is also preferably substantially free of culture medium, i.e., culture medium represents less than about 20%, more preferably less than about 10%, and most preferably less than about 5% of the volume of the preparation of the protein of interest.

As used herein, a “gene” includes a polynucleotide containing at least one open reading frame that is capable of encoding a particular polypeptide or protein after being transcribed and translated. Any of the polynucleotide sequences described herein may also be used to identify larger fragments or full-length coding sequences of the gene with which they are associated. Methods of isolating larger fragment sequences are known to those of skill in the art. As used herein, a “naturally-occurring” polynucleotide molecule includes, for example, an RNA or DNA molecule having a nucleotide sequence that occurs in nature (e.g., encodes a natural protein).

In preferred embodiments, the cDNAs encoding proteins of interest that endogenously and/or naturally expressed in the model taste cells of the present invention are isolated from the model taste cells mRNA using RT-PCR method that is well known in the art. As used herein, the term “cDNAs” includes DNA that is complementary to mRNA molecules present in the model taste cells. mRNA that can be converted into cDNA with an enzyme such as reverse transcriptase.

As used herein, the terms “polynucleotide,” “nucleic acid/nucleotide” and “oligonucleotide” are used interchangeably, and include polymeric forms of nucleotides of any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, DNA, cDNA, genomic DNA, recombinant polynucleotides, branched polynucleotides, plasmids, vectors, isolated DNA of any sequence, isolated RNA of any sequence, nucleic acid probes, and primers. Polynucleotides may be naturally-occurring, synthetic, recombinant or any combination thereof A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, any embodiment of this invention that is a polynucleotide encompasses both the double-stranded form and each of two complementary single-stranded forms known or predicted to make up the double-stranded form.

As used herein, the term “polynucleotide sequence” is the alphabetical representation of a polynucleotide molecule. A polynucleotide is composed of a specific sequence of four nucleotide bases: adenine (A); cytosine (C); guanine (C); thymine (T); and uracil (U) in place of thymine when the polynucleotide is RNA This alphabetical representation can be inputted into databases in a computer and used for bioinformatics applications such as, for example, functional genomics and homology searching.

As used herein, the term “isolated polynucleotide/cDNA molecule” includes polynucleotide molecules which are separated from other polynucleotide molecules which are present in the natural source of the polynucleotide. For example, with regard to genomic DNA, the term “isolated” includes polynucleotide molecules which are separated from the chromosome with which the genomic DNA is naturally associated. Preferably, an “isolated” polynucleotide is free of sequences which naturally flank the polynucleotide (i.e., sequences located at the 5′ and 3′ ends of the polynucleotide of interest) in the genomic DNA of the organism from which the polynucleotide is derived. For example, in various embodiments, the isolated polynucleotide molecule of the invention, or polynucleotide molecule encoding a polypeptide of the invention, can contain less than about 5 kb, 4 kb, 3 kb, 2 kb, 1 kb, 0.5 kb or 0.1 kb of nucleotide sequences which naturally flank the polynucleotide molecule in genomic DNA of the cell from which the polynucleotide is derived. Moreover, an “isolated” polynucleotide molecule, such as a cDNA molecule, can be substantially free of other cellular material, or culture medium when produced by recombinant techniques, or substantially free of chemical precursors or other chemicals when chemically synthesized.

As used herein, the term “polypeptide” or “protein” is interchangeable, and includes a compound of two or more subunit amino acids, amino acid analogs, or peptidomimetics. The subunits may be linked by peptide bonds. In another embodiment, the subunit may be linked by other bonds, e.g., ester, ether, etc. As used herein, the term “amino acid” includes either natural and/or unnatural or synthetic amino acids, including glycine and both the D or L optical isomers, and amino acid analogs and peptidomimetics. A peptide of three or more amino acids is commonly referred to as an oligopeptide. Peptide chains of greater than three or more amino acids are referred to as a polypeptide or a protein.

In preferred embodiments, the proteins of interest that endogenously and/or naturally expressed in the model taste cells of the present invention necessary for taste signaling also include proteins encoded by polynucleotides that hybridize to the polynucleotide encoding the proteins of interest under stringent conditions. As used herein, “hybridization” includes a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under different stringent conditions. The present invention includes polynucleotides capable of hybridizing under reduced stringency conditions, more preferably stringent conditions, and most preferably highly stringent conditions, to polynucleotides encoding the proteins of interest described herein. As used herein, the term “stringent conditions” refers to hybridization overnight at 60° C. in 10× Denhart's solution, 6×SSC, 0.5% SDS, and 100 μg/ml denatured salmon sperm DNA. Blots are washed sequentially at 62° C. for 30 minutes each time in 3×SSC/0.1% SDS, followed by 1×SSC/0.1% SDS, and finally 0.1×SSC/0.1% SDS. As also used herein, in a preferred embodiment, the phrase “stringent conditions” refers to hybridization in a 6×SSC solution at 65° C. In another embodiment, “highly stringent conditions” refers to hybridization overnight at 65° C. in 10× Denhart's solution, 6×SSC, 0.5% SDS and 100 μg/ml denatured salmon sperm DNA. Blots are washed sequentially at 65° C. for 30 minutes each time in 3×SSC/0.1% SDS, followed by 1×SSC/0.1% SDS, and finally 0.1×SSC/0.1% SDS. Methods for nucleic acid hybridizations are described in Meinkoth and Wahl, 1984, Anal. Biochem. 138:267-284; Current Protocols in Molecular Biology, Chapter 2, Ausubel et al., eds., Greene Publishing and Wiley-Interscience, New York, 1995; and Tijssen, 1993, Laboratory Techniques in Biochemistry and Molecular Biology: Hybridization with Nucleic Acid Probes, Part I, Chapter 2, Elsevier, New York, 1993. In one preferred embodiment, the proteins of interest encoded by nucleic acids used herein include nucleic acid having at least 60% homologous, preferably 75% homologous, more preferably 85%, more preferably 90%, most preferably 95%, 96%, 97%, 98%, 99% homologous to the polynucleotide sequences encoding the proteins of interest. In another preferred embodiment, the present invention also provides the model taste cells that endogenously and/or naturally express proteins having at least 60% homologous, preferably 75% homologous, more preferably 85%, more preferably 90%, most preferably 95%, 96%, 97%, 98%, 99% homologous to the amino acid sequences of the proteins of interest.

Moreover, the proteins of interest used herein can also be chimeric protein or fusion protein. As used herein, a “chimeric protein” or “fusion protein” comprises a first polypeptide operatively linked to a second polypeptide. Chimeric proteins may optionally comprise a third, fourth or fifth or other polypeptide operatively linked to a first or second polypeptide. Chimeric proteins may comprise two or more different polypeptides. Chimeric proteins may comprise multiple copies of the same polypeptide. Chimeric proteins may also comprise one or more mutations in one or more of the polypeptides. Methods for making chimeric proteins are well known in the art. In one embodiment of the present invention, the chimeric protein is a chimera of one taste receptor protein with another taste receptor proteins. In yet another embodiment of the present invention. the chimeric protein can be a chimera of one G protein with another G protein, or a chimera of one subunit of G protein with another subunit of G protein.

In yet another preferred embodiment, the present invention provides methods of screening for a plurality of compounds for enhancing sweet taste and/or inhibiting bitter taste. Such methods comprise 1) providing the model taste cells of the present invention, wherein the model taste cells endogenously and/or naturally express sweetener receptors and one or more other proteins and/or the relevant cellular machinery necessary for taste signaling; 2) contacting the model taste cells with a tastant alone, or in combination with test compounds; 3) determining effects of test compounds on the model taste cells using cell-based assays to monitor one or more of a) changes in intracellular second messengers (e.g., cAMP, cGMP, calcium, phophoinositides); b) changes in protein kinase activity (e.g., ERK, PKC, Src, EGFR, etc.); c) changes in gastrointestinal peptide secretion d) changes in neurotransmitter secretion by model taste cell; 4) identifying a compound that provide the changes as described above in 3); and 5) validating an efficacy of the identified compound in human sensory taste tests for modulating the taste by the tastant. As used herein, the “tastant” refers to any substances and/or compounds that are able to affect taste sensation. Such tastants include, but are not limited to, sweeteners, bitters, salty substances, sour substances, etc.

As used herein, the “sweetener” includes but is not limited to a) carbohydrate sweeteners including but not limited to sucrose, glucose, fructose, HFCS, HFSS, D-Tagatose, Trehalose, D-galactose, hamnose; b) synthetic high-potency sweeteners including but not limited to aspartame, neotame, acesulfame K, sucralose, cyclamate, saccharin, neohesperidindihydrochalcone; c) natural high-potency sweeteners including but not limited to, rebaudioside A, Rebaudioside B, Rebaudioside C, Rebaudioside D, Rebaudioside E, Dulcoside A, Dulcoside B, Rubusoside, Stevioside, Mogroside IV, Mogroside V, Monatin, Curculin, Glycyrrhizin, Thaumatin, Monellin, Mabinlin, Brazzein, Monatin, Hernandulcin, Phyllodulci; d) polyols including but not limited to Erythritol, Maltitol, Mannitol, Sorbitol, Lactitol, Xylitol, Isomalt, and e) amino acids including but not limited to Glycine, D- or L-alanine, D-tryptophan, arginine, serine, threonine.

In one preferred embodiment, the present invention provides cell-based assays for monitoring intracellular second messengers. In one preferred embodiment, the present invention provides an assay for measuring cyclic nucleotides, including cAMP and/or cGMP. In yet another preferred embodiment, the present invention provides assays for measuring intracellular calcium release. In yet another preferred embodiment, the present invention provides an assay for measuring phosphoinositides using traditional methods. In yet another preferred embodiment, the present invention provides cell-based assays for monitoring activities of protein kinases, such as serine/threonine kinases and ERK1 and 2. In yet another preferred embodiment, the present invention provides cell-based assays for monitoring neurotransmitter secretion from the model taste cells of the present invention. In yet another embodiment, the present invention provides cell-based assays for monitoring gastrointestinal peptide secretion from the model taste cells of the present invention.

In yet another preferred embodiment, the present invention provides methods of screening for a plurality of compounds for enhancing sweet taste. Such methods comprise providing the model taste cells of the present invention, wherein the model taste cells naturally express RGS proteins and one or more other proteins and/or the relevant cellular machinery necessary for sweetener signaling, such as Gα proteins; identifying compounds that inhibit RGS protein activity (RGS protein inhibitors); determining a sweet signaling activated by a sweetener receptor with a sweetener alone, and in combination with the compounds (RGS protein inhibitors); and identifying compounds (ROS protein inhibitors) that increase the sweet signaling of said sweetener. In one preferred embodiment, the RGS protein is RGS21 protein.

In particular, the effects of a sweetener as defined above on one of these signaling ‘readouts’ to the effects of the sweetener combined with an identified modulatory compound are compared in the model taste cells. In one preferred embodiment, the present invention provides that sweetener receptor activators, and/or a RGS21 protein inhibitor increases the observed effect of the sweetener. For instance, if the sweetener alone increases the release of intracellular calcium, then a combination of the sweetener with a sweetener receptor activator, and/or an RGS21 inhibitor should increase calcium release above the sweetener alone. In yet another preferred embodiment, the present invention also provides methods of screening for a plurality of compounds for modulating any taste sensations using the model taste cells of the present invention.

Moreover, the present invention provides methods to validate the effects of identified compounds and/or modulators in the model taste cells of the present invention on human sweet taste, as well as umami and bitter taste. In one preferred embodiment, the present invention provides a comparison of the perceived taste of a test tastant tasted by itself to that of a combination of a test tastant and the identified modulatory compounds.

The present invention also provides methods of conducting high-throughput screening for test compounds capable of inhibiting and/or modulating activity or expression of genes and/or proteins of interest in the model taste cells as defined above. A variety of high-throughput functional assays well-known in the art may be used in combination to screen and/or study the reactivity of different types of activating test compounds, but since the coupling system is often difficult to predict, a number of assays may need to be configured to detect a wide range of coupling mechanisms. A variety of fluorescence-based techniques is well-known in the art and is capable of high-throughput and ultra high-throughput screening for activity. The ability to screen a large volume and a variety of test compounds with great sensitivity permits analysis of the potential inhibitors and/or modulators for taste signaling.

The present invention provides methods for high-throughput screening of test compounds for the ability to modulate activity of genes and/or proteins of interest, and/or their interaction with other proteins in taste signaling transduction using the model taste cells, by combining the test compounds and the gene and/or protein of interest in high-throughput assays or in fluorescence based assays as known in the art. In one embodiment, the high-throughput screening assay detects the ability of a plurality of test compounds to bind to taste receptor genes and/or proteins. In another embodiment, the high-throughput screening assay detects the ability of a plurality of a test compound to inhibit a RGS protein binding partner (such as Gα protein) to bind to RGS protein. In yet another embodiment, the high-throughput screening assay detects the ability of a plurality of a test compounds to modulate taste signaling through taste receptor signaling transduction.

The present invention further provides a composition comprising inhibitors and/or modulatory compounds of genes and/or proteins of interest in the model taste cells for enhancing sweet taste signaling. The present invention also provides a composition comprising inhibitors and/or modulatory compounds of genes and/or protein of interest in the model taste cells for modulating umami and bitter taste, other than just sweet taste.

These and many other variations and embodiments of the invention will be apparent to one of skill in the art upon a review of the appended description and examples.

### EXAMPLES

### Example 1

### Methods for Producing the Model Taste Cells

Human HuTu-80 endocrine cells are produced based on the methods described by Rozengurt et al. (2006, Am J Physiol Gastrointest Liver Physiol 291:792-802). The parental HuTu-80 (ATCC: HTB-40™) endocrine cells are grow in minimum essential Eagle's medium containing 10% FBS and antibiotics (100 U/ml penicillin, 100 μg/ml streptomycin, and 0.25 μg/ml amphotericin B) in plastic or collagen I-coated plates in 5% CO₂/95% air at 37° C.

### Example 2

### Screening and Identifying Modulators for Taste Signaling Using the Model Taste Cells

One or more proteins of interest necessary for taste signal transduction are isolated and purified from the model taste cells as described above. The effects of test compounds on the purified proteins of interest or their interactions with other proteins in taste signal transduction are determined using variety of cell-based assays described below. The test compound that modulate the purified proteins of interest, or their interactions with other proteins in taste signal transduction based on the cell-based assays performed is identified, and then further validated in modulating the taste signaling.

Cell-based assays used herein include:

### A. Cell-Based Assays of Intracellular Second Messensgers:

Measurement of cyclic nucleotides: Changes in cyclic nucleotides such as cAMP and cGMP can be measured by quantifying their amounts in cell extracts by using a commercially available non-radioactive Alphascreen CAMP assay (Perkin-Elmer). The Alphascreen cAMP assay has been designed to directly measure levels of cAMP produced upon modulation of adenylate cyclase activity by GPCRs. The assay is based on the competition between endogenous cAMP and exogenously added biotinylated cAMP. The capture of cAMP is achieved by using a specific antibody conjugated to Acceptor beads. The assay is efficient at measuring both agonist and antagonist activities on Gαi- and Gαs-coupled GPCRs. The T1R and T2R family of GPCRs activate gustducin, which is a Gαi family G protein.

HuTu-80 cells are plated in multi-well plates in stimulation buffer, pH 7.4, (PBS containing 0.5 mM IBMX, 5 mM HEPES, 0.1% BSA) and anti-cAMP antibody conjugated acceptor beads. The cells are then treated with an empirically-determined concentration of forskolin to produce cAMP at 50% of their maximal capacity over 30 min. Varying concentrations of a tastant (e.g., sucrose, aspartame, etc.) is added along with forskolin and a putative taste modulatory compound. The cells are incubated for 30 min in the dark and then incubated with a mixture of streptavidin-coated beads bound to biotinylated cAMP (0.25 U/μl) in cell lysis buffer for 4 hr in the dark. The fluorescence signal is measured in a Perkin-Elmer Envision plate reader. In this experimental system, increasing concentrations of tastants are expected to increase the Alphascreen signal due to inhibition of adenylyl cyclase, which decreases the cellular cAMP available for competition with the biotinylated cAMP and the anti-cAMP antibody beads.

Alternatively, the model taste cells may be stably transfected with plasmid DNA that expresses a transcriptional reporter protein (e.g., luciferase, β-galactosidase, etc.) in proportion to the amount of cAMP; this assay monitors the activation of the cAMP-sensitive transcription factor, cAMP response element binding protein (CREB).

HuTu-80 cells are plated in 24-well plates and co-transfected with a CRE-luciferase (firefly) reporter plasmid (0.4 μg) and with pRL-Tk (0.1 μg), which constitutively expresses Renilla luciferase as a control for transfection efficiency, using Lipofectamine reagent (Invitrogen) as described (Nguyen et al., 2004, Cellular Signaling 16:1141-1151; Lee et al., 2004, Mol. Endocrin. 18:1740-1755). The cells are then treated with an empirically-determined concentration of forskolin in PBS containing 10 mM HEPES and 0.1% BSA, pH 7.4 to produce cAMP at 50% of their maximal capacity over 5-12 hr. Varying concentrations of a tastant (e.g., sucrose, aspartame, etc.) is added along with the forskolin and a putative taste modulatory compound for 5-12 hr. The cells are solubilized and the activities of the firefly luciferase and Renilla luciferase are determined using a commercially available Dual Luciferase assay kit (Promega) as per manufacturer's instructions. The firefly luciferase activity is divided by the Renilla luciferase activity to normalize for variations in transfection efficiency and is plotted as a function of the log₁₀ of the concentration of tastant.

Measurement of intracellular calcium: Changes in intracellular calcium can be measured in whole model taste cells by monitoring changes in fluorescence intensity and emission of calcium sensitive dyes (e.g., FURA-2, Fluo-3, etc.); these dyes are commercially available. Briefly, HuTu-80 cells are grown in 96-well plates for 24 hr and then rinsed twice with Hanks' balanced salt solution (GIBCO-BRL) supplemented with HEPES (pH 7.4), 1.26 mM CaCl₂, 0.5 mM MgCl₂, 0.4 mM MgSO₄, and 0.1% BSA (referred to as Ca⁺⁺ buffer) and were incubated at 37° C. for 15 min in 1 ml of the same buffer with 1.0 μM fura 2-AM. Cultures were then washed three times with Ca⁺⁺ buffer, and incubated with varying concentrations of tastants (e.g., sucrose, denatonium, etc.) in the presence or absence of a putative taste modulatory compound for 20 to 30 sec prior to averaging the fluorescence responses (480-nm excitation and 535-nm emission) in a Perkin-Elmer fluorescence plate reader. The data is corrected for background fluorescence measured before compound addition, and then normalized to the response to the calcium ionophore, ionomycin (1 μM, Calbiochem).

Alternatively, changes in intracellular calcium release can be measured by transfecting HuTu-80 cells with a plasmid that encodes the calcium-sensing fluorescent protein, Aequorin, whose fluorescence emission is increased upon binding to calcium in the presence of the substrate, coelenterazine. The affinity of aequorin to calcium is in the low micromolar range, and the activity of the enzyme is proportional to calcium concentration in the physiological range (50 nM to 50 μM) (Brini et al., J. Biol. Chem. 270: 9896-9903, 1995; Rizzuto et al., Biochem. Biophys. Res. Commun. 126: 1259-1268, 1995).

Measurement of Phosphoinositides by traditional approaches: Sweeteners lead to the activation of the enzyme PLC-β₂ in the model taste cells. This enzyme cleaves phosphatidylinositol bisphosphate (PIP₂) into the second messengers, inositol trisphosphate (IP₃) and diacylglycerol (DAG). Changes in PIP₂ can be monitored by quantifying the hydrolysis of radioactively labeled phosphoinositides using anion exchange chromatographies (Paing et al., 2002, J. Biol. Chem. 277:1292-1300).

HuTu-80 cells are labeled for 24 h with [³H]-labeled myo-D-inositol and the cell medium is replaced with 10 mM HEPES buffer, and 20 mM lithium chloride containing 1 mg/ml BSA. Cells are then stimulated with a tastant for up to 30 min at 37° C., extracted with 50 mM formic acid for 45 min at room temperature, and then neutralized with 150 mM NH₄OH. Cell extracts were then loaded directly on anion-exchange AG 1-X8 resin (100-200 mesh size, Bio-Rad) columns, washed with H₂O and then 50 mM ammonium formate, and eluted with 1.2 M ammonium formate, 0.1 M formic acid. Inositol mono-, bis-, and triphosphates eluted in this assay are quantified by scintillation counting.

Alternatively, the production of IP₃ can be measured using an IP₃ alphascreen assay, which is similar to the cAMP Alphascreen assay described above. The IP₃ alphascreen assay measures the ability of cellular IP₃, which is generated in response to sweetener receptor activation via PLC-β₂, to compete with biotinylated IP₃-beads to bind to acceptor beads that contain an IP₃ binding protein. Thus, increasing concentrations of sweeteners are expected to increase the cellular concentration of IP₃, which would then lead to a dose-dependent decrease in the alphascreen signal.

HuTu-80 cells in grown in 96-well plates are incubated with increasing concentrations of a tastants (e.g, sucrose, denatonium, etc.) in the presence or absence of a putative taste modulatory compound for 30 sec (in PBS/Hepes pH 7.4). The cells are then detergent solubilized and incubated with the alphascreen reagents as per manufacturer's instructions and the fluorescence signal is measured with a PerkinEilmer fluorescence plate reader.

### B. Cell-Based Assays of Protein Kinase Activities

Activation of the sweetener receptor has been shown to activate the serine/threonine kinases, ERKs 1 and 2, via a Gᵢ signaling pathway (Ozeck, et al., 2004, Eur. J. Pharm. 489:139-49). In addition to the ERKs, many other kinases are also activated via Gᵢ signaling pathways including serine/threonine kinases such as Akt and receptor tyrosine kinases such as the epidermal growth factor receptor (EGF-R) tyrosine kinase. A key step in the activation of many kinases, which can be experimentally determined, is the phosphorylation of the kinase itself. The most common way to determine the extent of activation of ERK1 and 2, for instance, is to use antibodies that are specific for the phosphorylated, and hence activated, form of ERK either by immunoassays or immunoblotting methods.

To measure the effects of sweetener receptor activation on the activity of ERK1, ERK2, Akt, MEK, and EGF-R, HuTu-80 cells grown in six-well dishes are treated with a tastants with or without a putative taste modulatory compound in PBS containing 10 mM HEPES and 0.1% BSA, pH 7.4 for 5-10 min at 37° C. and then solubilized in detergent buffer. Cell extracts from the treated cells are analyzed using anti-phospho kinase antibodies either in a plate immunoassay (Perkin-Elmer) or by immunoblotting. In addition, to analysis with phospho-specific antibodies, parallel samples of cell extracts will also be analyzed using antibodies that recognize total kinase (both phosphorylated and non-phosphorylated) by plate immunoassay (Perkin-Elmer) or immunoblotting. The ratio of phosphorylated kinase-to-total kinase is directly proportional to sweetener concentration.

### C. Measurement of Neurotransmitter Secretion

The final and most important step in taste cell signaling is the release of neurotransmitters, which further stimulate afferent nerve fibers. Finger and colleagues have shown that ATP is a critical ‘neurotransmitter’ that is secreted from taste cells and which interacts with specific purinergic, ATP-binding, receptors on nerve fibers (Finger et al., 2005, Science 310:1495-99).

HuTu-80 cells, grown in 96-well plates, are rinsed in PBS containing 10 mM HEPES and 0.1% BSA, pH 7.4 and stimulated with a tastant in the same buffer for 0-30 min at 37° C. Samples of the culture medium of stimulated HuTu-80 cells are collected and the concentration of ATP is determined using commercially available luminescence assay for ATP (e.g., ATPlite assay, Perkin-Elmer).

### D. Measurement of Gastrointestinal Peptide Secretion

Enteroendocrine cells such as HuTu-80 cells are known to secrete gastrointestinal peptides (e.g., peptide YY (PYY), glucagon, glucagon-like peptide-1 (GLP-1), gastric insulinotropic peptide (GIP), etc.) in response to taste receptor stimulation (Rozengurt, 2006, Am. J. Physiol Gastrointest Liver Physiol. 291: G171-G177). To measure secretion of GI peptides from HuTu-80 cells, competitive ELISA or RIA can be used. As an example, secretion of GLP-1 can be measured using commercially available competitive enzymatic immunoassays (e.g., Cosmo Bio Co., Ltd.).

Briefly, HuTu-80 cells are grown in multiwell dishes (e.g., 6-well, 12-well, etc.), are rinsed in PBS containing 10 mM HEPES and 0.1% BSA, pH 7.4, and stimulated with a tastant in the presence and absence of a test taste modulatory compound in the same buffer for 0-30 min at 37° C. Samples of the culture medium of stimulated HuTu-80 cells are collected and added to 96-well plates, which are coated with goat anti-GLP-1 antibodies, along with biotinylated GLP-1 standard, and rabbit anti-GLP-1 antibodies. The plates are incubated in the dark at 4° C. overnight for 16-18 hr. The well are rinsed with PBS, pH 7.4 and incubated with streptavidin-HRP for 1 hr at room temperature in the dark. After removing the streptavidin-HRP and rinsing with PBS, pH 7.4, o-phenylenediamine hydrochloride substrate solution is added and the reaction is developed in the dark for 30 min at room temperature. The reaction is stopped, and the optical absorbance of the wells is measured at 492 nm. The amount of secreted GLP-1 is determined by comparison to a standard curve, which is generated in parallel with known amounts of recombinant SLP-1.

### Example 3

### Validation of Effects of Taste Modulatory Compounds in Human Taste Tests

The perceived intensity of a test tastant (e.g., sweetener, savory compound, salty tastant, bitter, or sour tastant) tasted by itself to that of a combination of a test tastant and the taste modulatory compound is compared. A candidate taste enhancer enhances the perceived intensity of the test tastant, whereas a taste inhibitor decreases the perceived intensity of the test tastant.

In a particular embodiment, a panel of assessors is used to measure the intensity of a test tastant solution. Briefly described, a panel of assessors (generally 8 to 12 individuals) is trained to evaluate taste intensity perception and measure intensity at several time points from when the sample is initially taken into the mouth until 3 minutes after it has been expectorated. Using statistical analysis, the results are compared between samples containing additives and samples that do not contain additives. A decrease in score for a time point measured after the sample has cleared the mouth indicates there has been a reduction in tastant perception.

The panel of assessors may be trained using procedures well known to those of ordinary skill in the art. In a particular embodiment, the panel of assessors may be trained using the Spectrum.™. Descriptive Analysis Method (Meilgaard et al, Sensory Evaluation Techniques, 3.sup.rd edition, Chapter 11). Desirably, the focus of training should be the recognition of and the measure of the basic tastes; specifically, sweet, salty, sour, umami, and bitter. In order to ensure accuracy and reproducibility of results, each assessor should repeat the measure of the tastant intensity about three to about five times per sample, taking at least a five minute break between each repetition and/or sample and rinsing well with water to clear the mouth.

Generally, the method of measuring tastant intensity comprises taking a 10 mL sample into the mouth, holding the sample in the mouth for 5 seconds and gently swirling the sample in the mouth. Tastant intensity perceived is rated after 5 seconds, the sample is expectorated (without swallowing following expectorating the sample), the mouth is rinsed with one mouthful of water (e.g., vigorously moving water in mouth as if with mouth wash) and the rinse water is expectorated. The tastant intensity perceived is rated immediately upon expectorating the rinse water, waiting 45 seconds and, while waiting those 45 seconds, identifying the time of maximum perceived taste intensity and rating this intensity at that time (moving the mouth normally and swallowing as needed). Between samples take a 5 minute break, rinsing well with water to clear the mouth.

What is claimed is:

1. Model taste cells derived from human HuTu-80 enteroendocrine cells, or derivative cells thereof, which naturally or recombinantly express one or more signaling proteins useful for taste signal transduction and exhibit taste cell functionality.

2. The model taste cells of claim 1 wherein said signaling proteins are taste receptors comprising sweetener receptors, its hetero- or homo-oligomers, or combination thereof.

3. The model taste cells of claim 2, wherein said sweetener receptors are hetero-oligomeric T1R2/T1R3 sweetener receptors.

4. The model taste cells of claim 2, wherein said sweetener receptors are homo-oligomeric T1R3/T1R3 or T1R2/T1R2 sweetener receptors.

5. The model taste cells of claim 2, wherein said sweetener receptors are T1R3 sweetener receptors.

6. The model taste cells of claim 1, wherein said signaling proteins comprising all proteins selected from a group consisting of taste receptor proteins, proteins, regulator G protein signaling (RGS) proteins, or effectors, wherein said proteins are necessary for taste signal transduction.

7. The model taste cells of claim 1, wherein said taste signal transduction is sweet taste signal transduction.

8. The model taste cells of claim 1, wherein said taste signal transduction is bitter taste signal transduction.

9. The model taste cells of claim 1, wherein said taste signal transduction is umami taste signal transduction.

10. The model taste cells of claim 1, wherein said human HuTu-80 enteroendocrine cells comprise subcloned or modified cells derived from said HuTu-80 cells.

11. A method of screening for a compound that modulates taste signaling using a model taste cell of claim 1, said methods comprising:
a) isolating and purifying one or more proteins of interest useful for taste signal transduction from the model taste cells of claim 1;
b) determining effects of the test compound on the purified proteins of interest or their interactions with other proteins in a taste signal transduction pathway using variety of cell-based assays;
c) identifying the test compound that modulates the purified proteins of interest, or their interactions with other proteins in taste signal transduction based on said cell-based assays; and
d) validating the compound in modulating the taste signaling in said model taste cells.

12. The method of claim 11, wherein said protein is selected from the group consisting of taste receptors, G proteins, RGS proteins, effectors, and any cellular machinery for taste sensation.

13. The method of claim 11, wherein said protein is a sweetener receptor comprising T1R receptor family, its homo- or heteoro-oligomers.

14. The method of claim 11, wherein said protein is a G protein comprising Gα proteins selected from the group consisting of Gαi proteins, α-gustducin, Gαi2, and others.

15. The method of claim 11, wherein said protein is a RGS protein comprising GAIP, RGSz1, RGS1, RGS2, RGS3, RGS4, RGS5, RGS6, RGS7, RGS8, RGS9, RGS10, RGS11, RGS12, RGS13, RGS14, RGS16, RGS17, RGS21, D-AKAP1, p115RhoGEF, PDZ-RhoGEF, bRET-RGS, Axin, or mCONDUCTIN.

16. The method of claim 12, wherein said effectors are selected from the group consisting of phospholipase C (PLC), cAMP, cGMP, IP3, calcium (Ca²⁺) and other second messengers.

17. The method of claim 11, wherein said effect is determined through observations of cell-based assays selected from the group consisting of assays for measuring calcium (Ca²⁺) release, assays for cAMP, cGMP, PIP2/IP3, or other second messengers, assays for measuring secretion of GI peptides, and assays for measuring neurotransmitter secretion in said model taste cells.

19. The method of claim 11, wherein said test compounds are further validated using sensory taste testing in said model taste cells.

20. A method of screening for a plurality of compounds for enhancing sweet taste, said methods comprising:
1) providing the model taste cells of claim 1, wherein the model taste cells naturally express sweetener receptors and one or more other proteins or a relevant cellular molecule necessary for sweetener signaling;
2) contacting said model taste cells with a sweetener alone, or in combination with test compounds;
3) determining effects of test compounds on said model taste cells using cell-based assays to monitor one or more of
a) changes in intracellular second messengers (e.g., cAMP, cGMP, calcium, phophoinositides);
b) changes in protein kinase activity (e.g., ERK, PKC, Src, EGFR, etc.);
c) changes in model taste cell secretion of GI peptides; and
d) changes in neurotransmitter secretion by model taste cell;
4) identifying a compound that provide the changes as described above in 3); and
5) validating an efficacy of the identified compound in human sensory taste tests for enhancing sweet taste by the sweetener in said model taste cells.

21. The method of claim 20, wherein said sweetener comprises carbohydrate sweeteners, synthetic high-potency sweeteners, natural high-potency sweeteners, polyols, and amino acids.

22. A method of screening a plurality of compounds for enhancing sweet taste, said method comprising:
1) provide the model taste cells of claim 1, wherein said model taste cells naturally express RGS proteins and one or more other proteins necessary for sweetener signaling;
2) identifying compounds that inhibit RGS protein activity (RGS protein inhibitors);
3) determining a sweet signaling activated by a sweetener receptor with a sweetener alone, and in combination with the compounds (RGS protein inhibitors); and
d) identifying compounds (RGS protein inhibitors) that increase the sweet signaling of said sweetener.

23. The method of claim 22, wherein said RGS protein is an RGS21 protein.

24. A method of screening for a plurality of compounds for modulating taste sensation, said methods comprising:
1) providing the model taste cells of claim 1, wherein the model taste cells naturally express taste receptors of interest and one or more other proteins or a relevant cellular molecule necessary for taste signaling;
2) contacting said model taste cells with a tastant alone, or in combination with test compounds;
3) determining effects of test compounds on said model taste cells using cell-based assays to monitor one or more of
a) changes in intracellular second messengers (e.g., cAMP, cGMP, calcium, phophoinositides);
b) changes in protein kinase activity (e.g., ERK, PKC, Src, EGFR, etc.);
c) changes in model taste cell secretion of GI peptides; or
d) changes in neurotransmitter secretion by said model taste cell;
4) identifying a compound that provide the changes as described above in 3); and
5) validating an efficacy of the identified compound in human sensory taste tests for modulating taste sensation by said tastant in said model taste cells.
